(19) United States
(12) Patent Application Publication
(10) Pub. No.: US 2020/0147198 A1
(43) Pub. Date: May 14, 2020

(54) Novel Multivalent Polysaccharide-Protein Conjugate Vaccine Composition and Formulation Thereof

(71) Applicant: MSD Wellcome Trust Hiiieman Laboratories PVT., Ltd., New Delhi, Delhi (IN)

(72) Inventors: Davinder Gill, New Delhi, Delhi (IN); Sandeep Sharma, Uttar Pradesh (IN)

(21) Appl. No.: 16/625,246

(22) PCT Filed: Apr. 26, 2018
(86) PCT No.: PCT/IN2018/050253

§ 371 Date: Dec. 20, 2019

(30) Foreign Application Priority Data
Jun. 27, 2017 (IN) 201711022396

(51) Int. Cl. A61K 39/095; A61K 47/64

(52) U.S. Cl. CPC A61K 39/095; A61K 2039/6037; A61K 47/646; A61K 47/6415

(57) ABSTRACT

Novel multivalent polysaccharide-protein conjugate vaccine formulation. The formulation is liquid or lyophilized or a Liquid-Lyo combination pentavalent formulation of Neisseria meningitidis serogroup A, C, Y, W and X capsular polysaccharides (Men A, C, Y, W, X), each said polysaccharide being conjugated separately to tetanus toxoid (TT) carrier protein to obtain Men A, C, Y, W, X-TT conjugates, with one or more buffer and with or without an adjuvant along with pharmaceutically acceptable components/excipients.

### RELATED APPLICATIONS

This application is a US National stage entry of International Application No. PCT/IN2018/050253, which designated the United States and was filed on Apr. 26, 2018, published in English which claims priority under 35 U.S.C. § 119 to Indian Application No. 201711022396, filed Jun. 27, 2017. The entire teachings of the above applications are incorporated herein by reference.

### FIELD OF THE INVENTION

The present invention relates to novel polysaccharide-protein conjugate vaccine composition. More particularly, the present invention relates to a pentavalent conjugate vaccine formulation of Neisseria meningitidis serogroup A, C, Y, W and X capsular polysaccharides (Men A, C, Y, W, X) conjugated to tetanus toxoid (TT) carrier protein along with pharmaceutically acceptable components/excipients. The pentavalent conjugate vaccine formulation of the present invention is in liquid or lyophilized form or a combination thereof.

### BACKGROUND OF THE INVENTION

Vaccine manufacture and composition is complex and tightly regulated to ensure safety of the individuals and to maximize efficacy and stability. All vaccines contain an active component which generates the protective immune response. The vaccines also contain pharmaceutically acceptable additional components to enhance the stability of formulation and/or immunogenicity for strong protective immune response.

The World Health Organization recommends that countries with a moderate or high rate of disease or with frequent outbreaks of a vaccine preventable disease should routinely vaccinate. In countries with a low risk of disease, they recommend that high risk groups should be immunized.

Meningococcal disease is an acute, potentially severe illness caused by the bacterium Neisseria meningitidis. N. meningitidis (Meningococcus) is an aerobic gram-negative bacterium that has been serologically classified mainly into 13 serogroups A, B, C, D, 29E, H, I, K, L, W135, X, Y and Z. The grouping system is based on the capsular polysaccharides of the organisms.

It has been mentioned on the official website of the WHO that N. meningitidis is one of the most common causes of bacterial meningitis in the world and the only bacterium capable of generating large epidemics of meningitis. Major epidemics with incidence rates of up to 1000 cases per 100,000 inhabitants have been reported, particularly in sub-Saharan Africa.

N. meningitidis is transmitted by aerosol or direct contact with respiratory secretions of patients or healthy human carriers. The endemic disease occurs primarily in children and adolescents, with highest attack rates in infants aged 3-12 months, whereas in epidemics older children and young adults may be more involved. However, the rapid progression of meningococcal disease frequently results in death within 1-2 days after onset, the N. meningitidis infections can be prevented by vaccination.

Immunization is the only rational approach to control the meningitis disease. The most effective vaccines for N. meningitidis are polysaccharide-protein conjugate vaccines which are formed by the covalent attachment of activated protein to active polysaccharide. Polysaccharide undergoes some chemical modification which is known as “activation” process prior to attachment to a carrier protein because most of the native polysaccharides cannot be chemically efficiently linked to a carrier protein without activation.

There are many conjugation reactions that have been employed for covalently linking polysaccharides to proteins. Three of the more commonly employed methods include:

1) reductive amination, wherein the aldehyde or ketone group on one component of the reaction reacts with the amino or hydrazide group on the other component, and the C═N double bond so formed is subsequently reduced to C═N single bond by a reducing agent;

2) cyanylation conjugation, wherein the polysaccharide is activated either by cyanogen bromide (CNBr) or by 1-cyano-4-dimethylaminopyridinium tetrafluoroborate (CDAP) to introduce a cyanate group to the hydroxyl group, which forms a covalent bond to the amino or hydrazide group upon addition of the protein component; and

3) a carbodiimide reaction, wherein carbodiimide activates the carboxyl group on one component of the conjugation reaction, and the activated carboxyl group reacts with the amino or hydrazide group on the other component. These reactions are also frequently employed to activate the components of the conjugate prior to the conjugation reaction.

According to one publication, historically Serogroup A (MenA) has been the most common cause of epidemic disease in sub-Saharan Africa. Serogroups B and C are responsible for the majority of cases in developed-countries, with the remaining cases being caused by serogroups W135 and Y. Serogroup C cases have been observed recently in sub-Saharan Africa where the serogroup was not so prevalent historically. Serogroup X has also emerged in few countries in recent past. According to Xie O et al, “Emergence of serogroup X meningococcal disease in Africa: need for a vaccine”, Vaccine. 2013 Jun. 12;31(27):2852-6, Serogroup X meningococci have previously been considered a rare cause of sporadic meningitis, but during 2006-2010, outbreaks of serogroup X meningitis occurred in Niger, Uganda, Kenya, Togo and Burkina Faso, the latter with at least 1300 cases of serogroup X meningitis among the 6732 reported cases.

According to another NCBI publication, in Togo during 2006-2009, serogroup X meningococci accounted for 16% of the 702 confirmed bacterial meningitis cases. Kozah district experienced a serogroup X meningococci outbreak in March 2007 with a serogroup X meningococci seasonal cumulative incidence of 33/100,000. In Burkina Faso during 2007-2010, serogroup X meningococci accounted for 7% of the 778 confirmed bacterial meningitis cases, with an increase from 2009 to 2010 (4% to 35% of all confirmed cases, respectively). In 2010, serogroup X meningococci epidemics occurred in northern and central regions of Burkina Faso; the highest district cumulative incidence of serogroup X meningococci was estimated as 130/100,000 during March-April.

Based on the above facts, a pentavalent meningococcal ACYWX polysaccharide-protein conjugate vaccine could offer broader coverage against meningococcal disease except serogroup B for which a conjugate vaccine is remote possibility due to structural similarity of MenB capsular polysaccharide with the molecules found in human neuronal cells. Currently, various monovalent or multivalent vaccines including serogroup A, C, Y and W135 polysaccharide conjugates are licensed for sale in market. The existing state of art discloses vaccines for the prevention of various serogroups including N. meningitidis serogroup A, B, C, W and Y such as the US patent application no. US2015/0044253 which discloses the immunogenic composition comprising a saccharide fragment obtained from Haemophilus influenzae serotype B (Hib), Neisseria meningitidis serogroup A, B, C, W, Y conjugated with a carrier protein. However, in the US2015/0044253, the carbamate linker is linked directly onto amino groups of the carrier protein which results into low conjugation efficiency.

Indian patent application 281/MUM/2012 discloses a lyophilized, pentavalent N. meningitidis polysaccharide-protein conjugate composition. However, the application is restricted to specific Men X strains and uses multiple carrier proteins.

PCT/EP2006/006188 teaches an immunogenic composition comprising meningococcal capsular polysaccharide from at least one of the groups A, C, W135 and Y conjugated to a carrier protein however it does not teach conjugate of Men X in the immunogenic composition.

The available multivalent meningococcal conjugate vaccines are of higher cost and have complex formulations. None of the prior art teaches N. meningitidis polysaccharide-protein conjugate formulation with Men X conjugates in liquid or lyophilized form or a combination thereof using an optimized combination of conjugation chemistry.

There is an urgent need of meningococcal conjugate vaccine which has higher homogeneity, higher immunogenicity, lower cost and is simple to formulate and administer.

### OBJECT OF THE INVENTION

In order to obviate the drawbacks in the existing state of art, the main object of present invention is to provide a novel polysaccharide-protein conjugate vaccine composition.

Another object of the present invention is to provide a novel polysaccharide-protein conjugate vaccine composition wherein the conjugates are produced using optimized combination of conjugation chemistry.

Yet another object of the present invention is to provide novel polysaccharide-protein conjugate vaccine composition wherein said composition is capable of being used in production of a pentavalent meningococcal ACYWX-TT combination vaccine containing serogroup ACYWX polysaccharides each conjugated to tetanus toxoid as carrier protein.

Yet another object of the present invention is to provide a novel vaccine formulation of polysaccharide-protein conjugate vaccine composition comprising of polysaccharide-protein conjugates along with pharmaceutically acceptable components/excipients.

Yet another object of the present invention is to provide a novel vaccine formulation of polysaccharide-protein conjugate vaccine composition comprising of pentavalent meningococcal ACYWX-TT polysaccharide-protein conjugates along with pharmaceutically acceptable components/excipients.

Yet another object of the present invention is to provide novel polysaccharide-protein conjugate vaccine formulation wherein said pentavalent meningococcal ACYWX-TT formulation is a liquid or lyophilized vaccine formulation or as a combination of lyophilized and liquid portions.

Yet another object of the present invention is to provide novel pentavalent meningococcal ACYWX polysaccharide-tetanus toxoid carrier protein conjugate vaccine composition and formulation wherein said composition has novel combination of conjugates prepared using optimized conjugation chemistries.

Yet another object of the present invention is to provide the optimum dosage of each of the conjugates in the vaccine composition and formulation.

Yet another object of the present invention is to provide a pentavalent vaccine formulation which is stable at high temperatures and shows high immunogenicity and antigenicity.

### SUMMARY OF THE INVENTION

Accordingly, the present invention provides a novel polysaccharide-protein conjugate vaccine composition and formulation thereof. More particularly, the present invention relates to a conjugate vaccine formulation comprising of polysaccharide-protein conjugates produced using conjugation chemistry. The conjugation chemistry used includes one or more out of but not limited to carbamate chemistry and/or cyanylation chemistry. The formulation of present invention is capable of being used in production of pentavalent meningococcal combination vaccines.

The polysaccharides used for preparing conjugates of the present invention are obtained through optimized fermentation process.

The novel vaccine formulation of polysaccharide-protein conjugate vaccine composition comprises of polysaccharide-protein conjugates along with pharmaceutically acceptable components/excipients. All the conjugates in vaccine composition and formulation have same carrier protein. The present invention provides a pentavalent vaccine formulation.

The novel polysaccharide-protein conjugate vaccine formulation of present invention comprises of five polysaccharide-protein conjugates. The polysaccharide is selected from gram negative bacteria, belonging to Neisseria meningitidis capsular serogroup A, C, Y, W and X polysaccharides. Tetanus toxoid is used as the carrier protein in preparing all the conjugates.

The capsular polysaccharide is degraded to smaller sizes suitable for conjugation with carrier protein to obtain conjugates with high antigenicity and high immunogenicity. The capsular polysaccharide is degraded in the size range of 0.38±0.1 Kd when tested for molecular size distribution using HPLC PWXL4000 and 5000 columns in series. The capsular polysaccharides are preferably in the size range of 0.38±0.06 Kd. Both polysaccharide and carrier protein are activated before conjugation. The conjugates are obtained with hydrazine or adipic acid dihydrazide as linker.

The sized degraded capsular polysaccharide (sized capsular polysaccharide) is conjugated with tetanus toxoid carrier protein. The formulation contains the conjugates prepared by optimized carbamate chemistry for one or more serogroups more preferably for serogroup X polysaccharide and cyanylation chemistry for one or more serogroups preferably for each of serogroup A, C, Y and W polysaccharides.

The pharmaceutically acceptable excipients can be adjuvant, buffer, preservative, stabilizer, surfactant, either alone or in combination. The formulation of present invention is liquid or lyophilized or a combination of liquid and lyophilized formulation (liquid-lyo formulation) with mono- or multi-dose regimen with or without a preservative. The present invention also provides the optimum dosage of each of the conjugates in the vaccine composition and formulation.

### BRIEF DESCRIPTION OF DRAWINGS

FIG. 1 a-e depicts Real Time Stability Studies on liquid Pentavalent Meningococcal ACYWX-TT Conjugate Vaccine at 5±3° C.

FIG. 2 a-e depicts Accelerated Stability Studies on liquid Pentavalent Meningococcal ACYWX-TT Conjugate Vaccine at 25±2° C.

FIG. 3 a-e: Stress Stability Studies on liquid Pentavalent Meningococcal Conjugate Vaccine at 37±2° C.

FIG. 4 a-e: depicts Post 2-dose anti-meningococcal mouse IgG and SBA titers against adjuvanted and non-adjuvanted liquid pentavalent meningococcal ACYWX-TT conjugate vaccine in comparison to licensed ACYW conjugate vaccine and/or vehicle control.

FIG. 5 a-e: depicts Post 2-dose anti-meningococcal rabbit IgG and SBA titers against Adjuvanted and non-adjuvanted liquid pentavalent meningococcal ACYWX-TT conjugate vaccine in comparison to licensed ACYW conjugate vaccine or vehicle control.

FIG. 6 a-e: Post 3-dose anti-meningococcal mouse IgG and SBA titers against adjuvanted liquid pentavalent meningococcal ACYWX-TT conjugate vaccine in comparison to a VVM7 adjuvanted liquid pentavalent vaccine and licensed ACYW conjugate vaccine or vehicle control.

### DETAILED DESCRIPTION OF INVENTION WITH NON-LIMITING EXAMPLES AND ILLUSTRATIONS

Accordingly, the present invention provides a novel polysaccharide-protein conjugate vaccine composition and formulation thereof. More particularly, the present invention relates to a conjugate vaccine composition comprising of polysaccharide-protein conjugates produced using conjugation chemistry. The conjugation chemistry used includes combination of optimized carbamate chemistry and cyanylation chemistry. The composition of present invention is capable of being used in production of a pentavalent combination vaccine.

The polysaccharides used for production of conjugate of the present invention are obtained through an animal component free fermentation process.

The novel vaccine formulation of present invention comprises of polysaccharide-protein conjugates along with pharmaceutically acceptable components/excipients. All the conjugates in vaccine composition and formulation have same carrier protein. The present invention provides a pentavalent vaccine formulation.

The novel polysaccharide-protein conjugate vaccine composition and formulation of present invention comprises of five individual polysaccharide-protein conjugates. The polysaccharide is selected from gram negative bacteria Neisseria meningitidis serogroup A, C, Y, W and X capsular polysaccharides. The carrier protein used for preparing all the conjugates is Tetanus Toxoid (TT).

The capsular polysaccharide is degraded to smaller sizes suitable for conjugation with carrier protein to obtain conjugates with high antigenicity, high immunogenicity and high stability. The capsular polysaccharide is degraded in the size range of 0.38±0.1 Kd, preferably 0.38±0.06 Kd when tested for molecular size distribution using HPLC PWXL4000 and 5000 columns in series. The polysaccharide and carrier protein are activated before conjugation. The conjugates are produced having linker arm between polysaccharide and protein moities. The linker is attached to either polysaccharide or carrier protein or both the polysaccharide and protein.

The novel multivalent polysaccharide-protein conjugate vaccine formulation of the present invention comprises of meningococcal serogroups A, C, Y, W, X polysaccharides each individually conjugated to tetanus toxoid (Men ACYWX-TT) wherein serogroup X polysaccharide is conjugated to tetanus toxoid using optimized carbamate chemistry and serogroup A, C, Y and W polysaccharides are conjugated using optimized cyanylation chemistry. Each said conjugate has the protein to polysaccharide ratio between 0.3-1.0.

The novel multivalent polysaccharide-protein conjugate vaccine formulation of the present invention comprises of meningococcal serogroups A, C, Y, W, X polysaccharides each individually conjugated to tetanus toxoid (Men ACYWX-TT) mixed with one or more buffer and one or more pharmaceutically acceptable excipients, with or without adjuvant.

The pharmaceutically acceptable excipients can be adjuvant, buffer, preservative, stabilizer, surfactant, either alone or in combination. The formulation of present invention is a liquid or lyophilized formulation or a liquid-lyo combination with mono- or multi-dose regimen with or without a preservative. The vaccine formulation of the present invention is stable at high temperatures. Considering 40% free PS as the maximum target, the formulation shows stability at the high temperature of 37±2° C. for at least 21 days, at 25±2° C. for at least 3 months and at 5±3° C. for at least 9 months.

In one of the best embodiments, the novel pentavalent liquid polysaccharide-protein conjugate vaccine formulation of the present invention comprises of:

| Ingredient | Quantity/Concentration |
|---|---|
| Men ACYWX-TT | 4-20 μg polysaccharide/serogroup/ml |
| Buffer | 5-30 mM Phosphate buffered saline (pH 7.0 ± 0.2) |
| Excipient | 0-150 mM NaCl sufficient to maintain osmolality |
| | between 240-330 mOsmol/Kg |
| | 5-30 mM Histidine |
| Adjuvant: | Aluminum phosphate as 500-2000 μg Al⁺⁺⁺/ml |
| Water (MQW) | qs |

The ingredients are mixed by stirring at room temperature for 0.5-2 hours and followed by filling in vials and storage at 2-8° C.

In another embodiment, the novel pentavalent liquid polysaccharide-protein conjugate vaccine formulation of the present invention comprises of:

| Ingredient | Quantity/Concentration |
|---|---|
| Men ACYWX-TT | 10-20 μg polysaccharide/serogroup/ml |
| Buffer | 10-25 mM Phosphate buffered saline (pH |
| | 7.0 ± 0.2) |
| Excipient | 0-150 mM NaCl to maintain osmolality between |
| | 240-330 mOsmol/Kg |
| Adjuvant: | Aluminum phosphate 750-1500 μgAl⁺⁺⁺/ml |
| Water (MQW) | qs |

The ingredients are mixed by stirring at room temperature for 0.5-2 hours and followed by filling in vials and storage at 2-8° C.

One of the best embodiments of the formulation with adjuvant providing desired osmolality and high stability and desired immunogenicity comprises of:

| Ingredient | Quantity/Concentration |
|---|---|
| Men ACYWX-TT | 10-20 μg polysaccharide/serogroup/ml |
| Buffer: | 10 mM PBS (pH 7.0 ± 0.2) |
| Excipient: | 0-150 mM NaCl |
| Adjuvant | Aluminum phosphate as 1000 μg Al⁺⁺⁺/ml |
| Water (MQW) | qs |

The ingredients are mixed by stirring at room temperature for 0.5-2 hours and followed by filling in vials and storage at 2-8° C.

Another embodiment of the formulation without adjuvant providing desired osmolality, high stability and desired immunogenicity comprises of

| Ingredient | Quantity/Concentration |
|---|---|
| Men ACYWX-TT | 10 μg polysaccharide/serogroup/ml |
| Buffer: | 10 mM PBS (pH 7.0 ± 0.2) |
| Excipients: | 0-150 mM NaCl |
| Water (MQW) | qs |

The ingredients are mixed by stirring at room temperature for 0.5-2 hours and followed by filling in vials and storage at 2-8° C.

The formulations of the present invention with or without adjuvant have been tested for osmolality, stability and immunogenicity. The formulations have been exposed to high temperatures of 37±2° C. (VVM) for 21 days to check the rise in the free polysaccharide content. The free polysaccharide content in the pentavalent vaccine formulation has been separated by deoxycholate (DOC) precipitation and filtration method and estimated by High performance anion exchange chromatography with pulsed amperometric detector (HPAEC-PAD) method.

The formulation is stable at high temperatures. Considering 40% free PS as the maximum target, the formulation shows stability at the high temperature of 37±2° C. for at least 21 days, at 25±2° C. for at least 3 months and at 5±3° C. for at least 9 months.

The formulation of the present invention is in liquid or lyophilized form or a combination thereof. The present invention also provides the optimum dosage of each of the conjugates in the vaccine composition and formulation. The optimum dose is 5-10 μg of serogroup A and X polysaccharide and 5 μg of serogroup C, Y and W polysaccharide per human dose without adjuvant or with 500 μg Al⁺⁺⁺ in form of aluminum phosphate adjuvant per human dose.

### Example 1: Preparation of Placebo Liquid Formulations

NaCl has been mixed with different buffers to achieve the osmolality of the buffer in the range of 300±30 mOsmol/Kg. A placebo formulation without any meningococcal serogroup (active antigen) has been prepared by mixing Aluminum phosphate with different Buffers and NaCl to know the osmotic strength of the formulation as per Table 1 below:

TABLE 1
Matrix for the preparation of placebo liquid formulation

| | | | | Alumimum |
|---|---|---|---|---|
| Formula- | | Strength | Strength | phosphate |
| tion No. | Buffer type | of NaCl | of buffer | content(as Al⁺⁺⁺) |
| MLF1 | Sodium chloride | 0.85% | — | 1 mg/ml |
| | (NaCl) | | | |
| MLF2 | 2-(N-morpholino) | 100 mM | 25 mM | 1 mg/ml |
| | ethanesulfonic | | | |
| | acid (MES) | | | |
| MLF3 | Phosphate | 100 mM | 25 mM | 1 mg/ml |
| | buffered saline | | | |
| | (PBS) | | | |
| MLF4 | L-Histidine | 100 mM | 25 mM | 1 mg/ml |
| MLF5 | MES & Histidine | 100 mM | 25 mM | 1 mg/ml |
| MLF6 | PBS & Histidine | 100 mM | 25 mM | 1 mg/ml |

The above formulations (placebo) have been analyzed for Osmolality and pH to determine the suitable concentration of NaCl to achieve the desired osmolality in the final formulations. The osmolality of the formulations is provided in Table 2.

TABLE 2
Osmolality and pH of the Placebo liquid formulations

| | Osmolality | |
|---|---|---|
| Formulation No. | (mOsmol/Kg) | pH |
| MLF1 | 417 | 6.64 |
| MLF2 | 357 | 6.77 |
| MLF3 | 367 | 6.97 |
| MLF4 | 346 | 6.69 |
| MLF5 | 352 | 6.78 |
| MLF6 | 353 | 6.85 |

Table 2 has suggested that the osmolality of the formulations of Table 1 has been higher than the expected limits of 240-330 mOsmol/Kg, indicating to reduce the strength of NaCl in order to reduce osmolality. The molar strength of NaCl has been reduced to achieve desired osmolality of the formulations.

### Example 2: Preparation of Placebo Liquid Formulation by Reducing the NaCl Strength

The NaCl strength has been reduced to half as compared to strength of NaCl in Table 1, i.e., form 100 mM to 50 mM to get the osmolality index within the desired range as per Table 3 below:

TABLE 3
Osmolality and pH of Placebo formulations

| | | | | Alumimum | | |
|---|---|---|---|---|---|---|
| | | | | phosphate | Results | |
| Formulation | Buffer | Strength | Strength | content | Osmolality | |
| No. | type | of NaCl | of buffer | (as Al⁺⁺⁺) | (mOsmol/Kg) | pH |
| MLF1 | NaCl | 0.42% | — | 1 mg/ml | 282 | 6.90 |
| MLF2 | MES | 50 Mm | 25 mM | 1 mg/ml | 275 | 6.62 |
| NaCl | — | 0.9% | — | — | 289 | 6.90 |
| Aluminum | — | — | — | 2 mg/ml | 276 | — |
| phosphate | | | | | | |

With NaCl close to 50 mM, the osmolality of the formulations achieved in the range of 275 mOsmol/Kg to 282 mOsmol/Kg which is within the range of desired osmolality for the formulations.

### Example 3: Preparation of Different Pentavalent Meningococcal Liquid Formulations to Establish the Product Stability in the Presence of Different Excipients and Buffers

Various pentavalent Meningococcal ACYWX-TT formulations have been prepared by mixing the antigen with the buffers like MES buffer, PBS buffer, Tris Buffer, HEPES buffer and Histidine. A combination of different excipients such as Glycine, Dextrose, Histidine, Mannitol and Polysorbate 80 have been tried to evaluate the stability of the drug product when exposed to a higher temperature of 37±2° C. The formulations have been prepared as per the Table 4 below:

TABLE 4
Matrix for the formulation of liquid pentavalent Meningococcal ACYWX-TT conjugate vaccine.

| | | | | | | | | | Antigen |
|---|---|---|---|---|---|---|---|---|---|
| | | NaCl | | | | | | Aluminum | 10 μg/ml |
| Formulation | | Total | Total | Histidine | Dextrose | Glycine | Tween 80 | phosphate | of each |
| Code | Buffer | 60 mM | 75 mM | 10 mM | 1% | 10 mM | 0.05% | 1 mg/ml | serogroup |
| MLF-7 | Normal | — | — | — | — | — | — | ✓ | ✓ |
| | saline | | | | | | | | |
| | 0.45% | | | | | | | | |
| | (pH 7.0) | | | | | | | | |
| MLF-13 | PBS | ✓ | — | — | — | — | — | ✓ | ✓ |
| MLF-14 | (pH 7.0) | — | ✓ | — | — | — | — | ✓ | ✓ |
| MLF-21 | MES | ✓ | — | — | — | — | — | ✓ | ✓ |
| MLF-22 | (pH 6.5) | — | ✓ | — | — | — | — | ✓ | ✓ |
| MLF-29 | Histidine | ✓ | — | — | — | — | — | ✓ | ✓ |
| | (pH 6.8) | | | | | | | | |
| MLF-30 | TRIS | ✓ | — | — | — | — | — | ✓ | ✓ |
| | (pH 7.0) | | | | | | | | |
| MLF-31 | HEPES | ✓ | — | — | — | — | — | ✓ | ✓ |
| | (pH 7.0) | | | | | | | | |
| MLF-32 | PBS/ | ✓ | — | ✓ | ✓ | ✓ | ✓ | ✓ | ✓ |
| | Hsitidine/ | | | | | | | | |
| | Dextrose/ | | | | | | | | |
| | Glycine/ | | | | | | | | |
| | Tween 80 | | | | | | | | |
| | (pH 7.0) | | | | | | | | |

The above formulations have been exposed to a temperature of 37±2° C. for 14 days to evaluate the effect of buffers and excipients in the stability profile of the product. The stability indicating parameter for Meningococcal conjugate formulations is the generation of free PS with time. The samples have been withdrawn on day 7 and day 14 and analyzed by a HPAEC-PAD (Dionex) method for total and free PS %. Results have been compiled for free Polysaccharide generated over time as per Table 5 and 6 below:

TABLE 5
Free Polysaccharide content in liquid pentavalent Meningococcal
ACYWX-TT liquid formulations for Men A, C & Y when
exposed to 37 ± 2° C. for up to 14 days.

| Formu- | Men A Free PS % | | | Men C Free PS % | | | Men Y Free PS % | | |
|---|---|---|---|---|---|---|---|---|---|
| lation | | 7 | 14 | | 7 | 14 | | 7 | 14 |
| Code | Initial | days | days | Initial | days | days | Initial | days | days |
| MLF-7 | <1 | 23 | 37 | 20 | 34 | 41 | 14 | 11 | 14 |
| MLF-13 | 2 | 6 | 8 | 22 | 29 | 31 | 11 | 13 | 14 |
| MLF-14 | <1 | 4 | 14 | 21 | 23 | 24 | 11 | 12 | 10 |
| MLF-21 | 3 | 12 | 24 | 20 | 32 | 38 | 10 | 10 | 13 |
| MLF-22 | 3 | 14 | 31 | 20 | 30 | 32 | 9 | 12 | 15 |
| MLF-29 | 2 | 7 | 15 | Int. | Int. | Int. | 14 | 11 | 12 |
| MLF-30 | 3 | 16 | 30 | 28 | 37 | 32 | Int. | Int. | Int. |
| MLF-31 | 2 | 20 | 35 | 19 | 25 | 42 | 9 | 17 | 12 |
| MLF-32 | Int. | Int. | Int. | 12 | 28 | 39 | Int. | Int. | Int. |

Int.: Assay interference

TABLE 6
Free Polysaccharide content in liquid pentavalent Meningococcal
ACYWX-TT conjugate vaccine formulations for Men W &
X when exposed to 37 ± 2° C. up to 14 days

| Formu- | Men W Free PS % | | | Men X Free PS % | | |
|---|---|---|---|---|---|---|
| lation | | 7 | 14 | | 7 | 14 |
| Code | Initial | days | days | Initial | days | days |
| MLF-7 | 18 | 18 | 20 | <1 | <1 | <1 |
| MLF-13 | 18 | 19 | 20 | <1 | <1 | <1 |
| MLF-14 | 18 | 19 | 21 | <1 | <1 | <1 |
| MLF-21 | 20 | 21 | 22 | <1 | <1 | <1 |
| MLF-22 | 19 | 21 | 22 | <1 | 3 | <1 |
| MLF-29 | 19 | 21 | 23 | <1 | 1 | <1 |
| MLF-30 | 18 | 18 | 18 | <1 | 2 | 1 |
| MLF-31 | 19 | 19 | 21 | <1 | <1 | 1 |
| MLF-32 | 17 | 18 | 21 | <1 | 1 | 3 |

### Example 4: Preparation of Different Pentavalent Meningococcal ACYWX-TT Liquid Formulations Without Aluminum Phosphate

The pentavalent Meningococcal formulations have been prepared without the addition of Aluminum phosphate. All the formulations have been prepared in PBS. The following matrix has been used to prepare the different formulations:

TABLE 7
Matrix for the preparation of liquid pentavalent meningococcal
ACYWX-TT conjugate vaccine formulations without adjuvant

| Formu- | | | | | | A, C, Y, W, |
|---|---|---|---|---|---|---|
| lation | | | Man- | Histi- | | X-TT (PS per |
| Code | PBS | NaCl | nitol | dine | MQW | conjugate) |
| MLF-36 | 10 mM | 100 mM | — | — | qs | 10 μg/ml |
| MLF-38 | 10 mM | 100 mM | — | 10 mM | qs | 10 μg/ml |
| MLF-39 | 10 mM | 100 mM | 0.1% | — | qs | 10 μg/ml |

The above formulations have been analyzed for osmolality and pH to confirm the basic characteristics of the formulation.

TABLE 8
Results of the liquid pentavalent meningococcal
conjugate vaccine formulations without adjuvant

| | Osmolality | |
|---|---|---|
| Formulation Code | (mOsmol/Kg) | pH |
| MLF-36 | 289 | 7.14 |
| MLF-38 | 315 | 7.32 |
| MLF-39 | 295 | 7.11 |

The results obtained meet the desired values of Osmolality and pH.

### Example 5: Preparation of Pentavalent Meningococcal ACYWX-TT Liquid Formulations with Higher Dose of Men A & Men X

The formulation has been prepared with higher dose of 10 μg/0.5 ml for serogroups MenA and MenX, while keeping dose of MenC, MenY, MenW at the dose of 5 μg/0.5 ml.

TABLE 9
Matrix for the preparation of formulations
with higher MenA and MenX dose

| Formula- | | | Aluminum | | A, C, Y, |
|---|---|---|---|---|---|
| tion | | | phosphate | | W, X-TT |
| Code | PBS | NaCl | (as Al⁺⁺⁺) | MQW | conjugates |
| MLF-35 | 10 mM | 100 mM | 500 μg/ml | qs | 20 μg/ml A &X |
| | | | | | and 10 μg/ml of |
| | | | | | C, Y &W |

The above formulation has been analyzed for polysaccharide content, Free polysaccharide %, Osmolality and pH to confirm the basic characteristics of the formulation.

TABLE 10
Results of the liquid pentavalent meningococcal ACYWX-TT
formulation having higher MenA & MenX dose

| | Osmolality | |
|---|---|---|
| Formulation Code | mOsmol/Kg | pH |
| MLF-35 | 321 | 7.0 |

TABLE 11
Results of the Liquid Pentavalent Meningococcal
ACYWX-TT Formulations having all serogroups
at 10 μg polysaccharide per ml

| Formulation | | Alum | Osmolality |
|---|---|---|---|
| Code | pH | mg/ml | mOsmol/Kg |
| MLF-13 | 7.06 | 0.84 | 320 |
| MLF-15 | 7.21 | 0.91 | 330 |
| MLF-33 | 7.02 | 1.04 | 326 |
| MLF-36 | 7.14 | — | 289 |
| MLF-38 | 7.32 | — | 315 |
| MLF-39 | 7.11 | — | 295 |

### Example 6: Preparation of Different Lyophilized Pentavalent Meningococcal Formulations to Establish the Product Stability in the Presence of Different Excipients and Buffers

Various pentavalent Meningococcal ACYWX-TT formulations have been prepared by mixing the antigen with the buffers like MES buffer and PBS buffer. A combination of different excipients such as Sucrose, Maltose, Arginine, Lactose, Sorbitol, Histidine, Glycine have been tried to evaluate the pH, osmolality and moisture content at room temperature and to achieve the desired moisture content after lyophilization.

The desired osmolality and pH has been achieved with 10 mM Sodium Phosphate buffer with different combinations of excipients (alone or in combination) wherein the moisture content after lyophilization is not more than 3% and the cake quality is satisfactory (Table 12).

TABLE 12
Analysis of various combinations for lyophilized
formulation (non-limiting examples):

| | | | | | | Moisture |
|---|---|---|---|---|---|---|
| Formulation | 10 mM Phosphate buffer plus | | | | Osmolality | content |
| code | Other Excipients | | | pH | (mOsmol/Kg) | (%) |
| MF59 | Sucrose | Lactose | Glycine | 6.5-7.5 | 240-330 | ≤3 |
| | (1%) | (4%) | (20 mM) | | | |
| MF60 | Sucrose | Maltose | Glycine | 6.5-7.5 | 240-330 | ≤3 |
| | (1%) | (4%) | (20 mM) | | | |
| MF61 | Sucrose | Mannitol | Glycine | 6.5-7.5 | 240-330 | ≤3 |
| | (1%) | (4%) | (20 mM) | | | |
| MF62 | Sucrose | Lactose | Glycine | 6.5-7.5 | 240-330 | ≤3 |
| | (2%) | (3%) | (20 mM) | | | |
| MF63 | Sucrose | Maltose | Glycine | 6.5-7.5 | 240-330 | ≤3 |
| | (2%) | (3%) | (20 mM) | | | |
| MF64 | Sucrose | Mannitol | Glycine | 6.5-7.5 | 240-330 | ≤3 |
| | (2%) | (3%) | (20 mM) | | | |
| MF65 | Sucrose | Lactose | Glycine | 6.5-7.5 | 240-330 | ≤3 |
| | (3%) | (2%) | (20 mM) | | | |
| MF66 | Sucrose | Maltose | Glycine | 6.5-7.5 | 240-330 | ≤3 |
| | (3%) | (2%) | (20 mM) | | | |
| MF67 | Sucrose | Mannitol | Glycine | 6.5-7.5 | 240-330 | ≤3 |
| | (3%) | (2%) | (20 mM) | | | |
| MF71 | Maltose | Lactose | Glycine | 6.5-7.5 | 240-330 | ≤3 |
| | (1%) | (4%) | (20 mM) | | | |
| MF72 | Maltose | Mannitol | Glycine | 6.5-7.5 | 240-330 | ≤3 |
| | (1%) | (4%) | (20 mM) | | | |
| MF74 | Maltose | Mannitol | Glycine | 6.5-7.5 | 240-330 | ≤3 |
| | 2%) | (3%) | (20 mM) | | | |
| MF75 | Maltose | Lactose | Glycine | 6.5-7.5 | 240-330 | ≤3 |
| | (3%) | (2%) | (20 mM) | | | |
| MF76 | Maltose | Mannitol | Glycine | 6.5-7.5 | 240-330 | ≤3 |
| | (3%) | (2%) | (20 mM) | | | |
| MF77 | Maltose | Lactose | Glycine | 6.5-7.5 | 240-330 | ≤3 |
| | (4%) | (1%) | (20 mM) | | | |
| MF82 | Lactose | Mannitol | Glycine | 6.5-7.5 | 240-330 | ≤3 |
| | (4%) | (1%) | (20 mM) | | | |

### Example 7: The Liquid-Lyo Combination Formulation

| Ingredient | Quantity/Concentration |
|---|---|
| Men A, C, Y, W, X -TT | 10-20 μg polysaccharide/serogroup/ml |
| Buffer: | 5-20 mM PBS (pH 6.5-7.5) |
| Excipients: | 2-10% |
| Diluent | qs |

In liquid-lyo formulation, the lyophilized (1yo) portion contains MenA-TT conjugate, MenC-TT conjugate either alone or in variable combinations, with excipient being selected from Sucrose, Maltose, Arginine, Lactose, Sorbitol, Histidine, Glycine, either alone or in variable combinations. The diluent is selected from water, 5-20 mM phosphate buffered saline, aluminum phosphate as 500-1500 containing MenC-TT, MenY-TT, MenX-TT, MenW-TT either alone or in variable combinations.

### Example 8: Lyophilization Cycles: (Non-Limiting Examples)

The pentavalent formulation has been lyophilized through freezing, primary drying and secondary drying. The lyophilization cycles are shown in Table 13. Lyophilization cycle-3 is the best mode yielding stable formulation with less than 3% moisture content and a good quality cake.

TABLE 13
Lyophilization cycles

| LYOPHILIZATION CYCLE-1 | | | | | LYOPHILIZATION CYCLE-2 | | | | | LYOPHILIZATION CYCLE-3 | | | | |
|---|---|---|---|---|---|---|---|---|---|---|---|---|---|---|
| Freezing | | | | | Freezing | | | | | Freezing | | | | |
| Step | Temp | Time | | R/H | Step | Temp | Time | | R/H | Step | Temp | Time | | |
| | (° C.) | (min) | | | | (° C.) | (min) | | | | (° C.) | (min) | | R/H |
| 1 | 4 | 60 | | H | 1 | 4 | 60 | | H | 1 | 4 | 60 | | H |
| 2 | −48 | 250 | | R | 2 | −48 | 250 | | R | 2 | −48 | 250 | | R |
| 3 | −48 | 5 | | H | 3 | −48 | 120 | | H | 3 | −48 | 360 | | H |
| Primary drying | | | | | Primary drying | | | | | Primary drying | | | | |
| Step | Temp | Time | Vacuum | R/H | Step | Temp | Time | Vacuum | R/H | Step | Temp | Time | Vacuum | |
| | (° C.) | (min) | (mTor) | | | (° C.) | (min) | (mTor) | | | (° C.) | (min) | (mTor) | R/H |
| 1 | −20 | 90 | 50 | R | 1 | −20 | 90 | 50 | R | 1 | −28 | 240 | 50 | R |
| 2 | −20 | 15 | 50 | H | 2 | −20 | 180 | 50 | H | 2 | −20 | 180 | 50 | R |
| 3 | 10 | 90 | 50 | R | 3 | 10 | 90 | 50 | R | 3 | −20 | 600 | 50 | H |
| 4 | 10 | 30 | 50 | H | 4 | 10 | 60 | 50 | H | 4 | −10 | 60 | 50 | R |
| 5 | 20 | 300 | 50 | R | 5 | 20 | 300 | 50 | R | 5 | 10 | 120 | 50 | R |
| 6 | 20 | 10 | 50 | H | 6 | 20 | 60 | 50 | H | 6 | 25 | 60 | 50 | R |
| 7 | 30 | 30 | 50 | R | 7 | 30 | 30 | 50 | R | 7 | 30 | 30 | 50 | R |
| Secondary drying | | | | | Secondary drying | | | | | Secondary drying | | | | |
| Step | Temp | Time | Vacuum | R/H | Step | Temp | Time | Vacuum | R/H | Step | Temp | Time | Vacuum | |
| | (° C.) | (min) | (mTor) | | | (° C.) | (min) | (mTor) | | | (° C.) | (min) | (mTor) | R/H |
| 1 | 30 | 810 | 50 | H | 1 | 30 | 810 | 50 | H | 1 | 30 | 500 | 50 | H |

R: Ramp;
H: Hold;
min: Minutes

### Example 9: Stability of Pentavalent Meningococcal ACYWX-TT Liquid Formulations at Stress, Accelerated and Real-Time Conditions

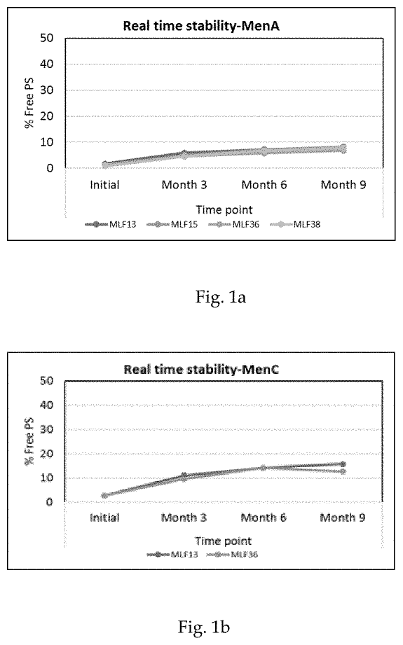

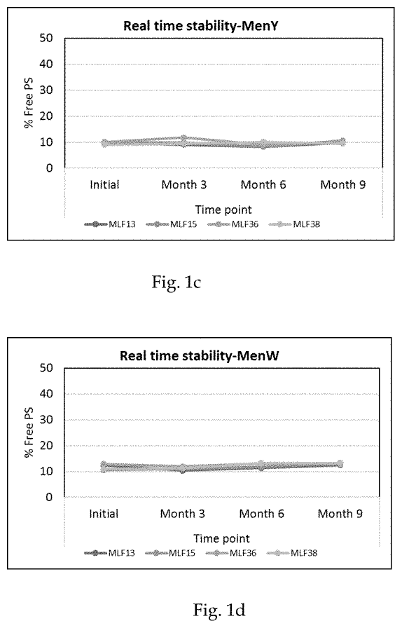

The stability of pentavalent meningococcal ACYWX-TT conjugate vaccine is tested when exposed at various temperature conditions over the period. The test has been conducted to get the effect of temperatures over certain periods of time. The stability tests have been conducted using three temperature parameters, viz. real-time storage conditions at 5±3° C., higher temperatures at 25±2° C. and stress conditions at 37±2° C. the results are mentioned in FIG. 1-3 (FIG. 1a-1e, 2a-2e, 3a-3e).

a. real-time storage conditions at 5±3° C.—recommended storage temperature (Real Time Stability Studies) (FIG. 1 a-e).

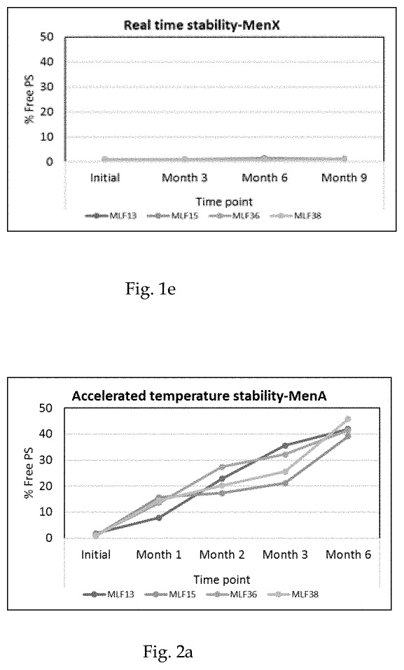

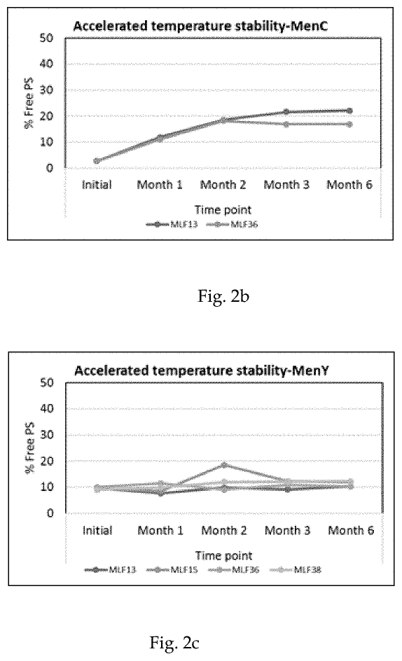

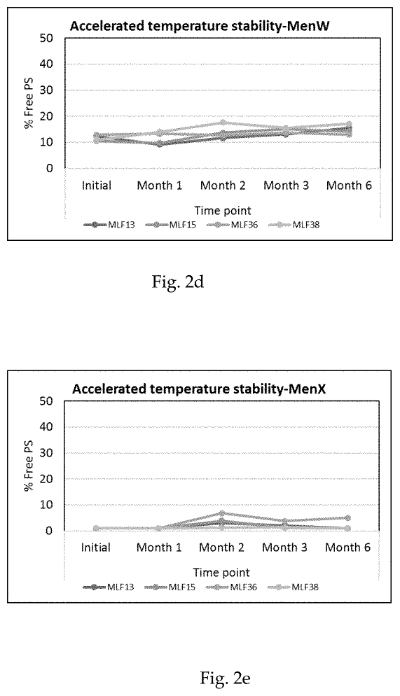

b. higher temperatures at 25±2° C.—higher than those recommended for storage (Accelerated Stability Studies) (FIG. 2 a-e).

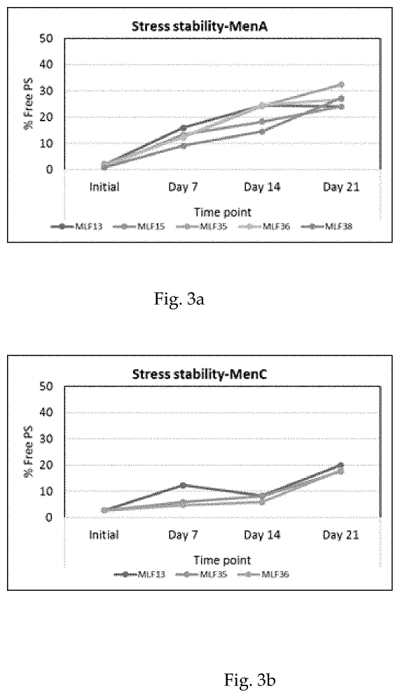

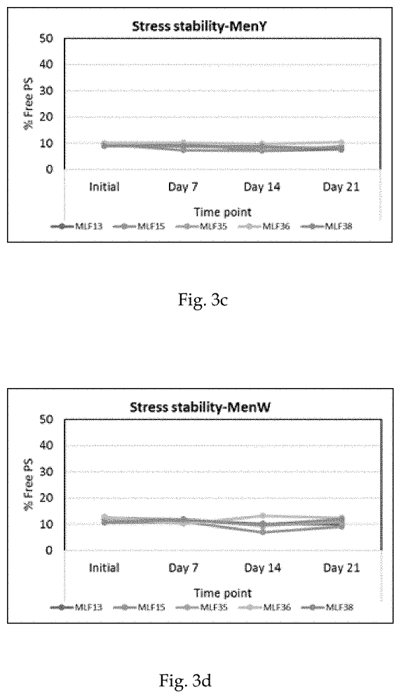

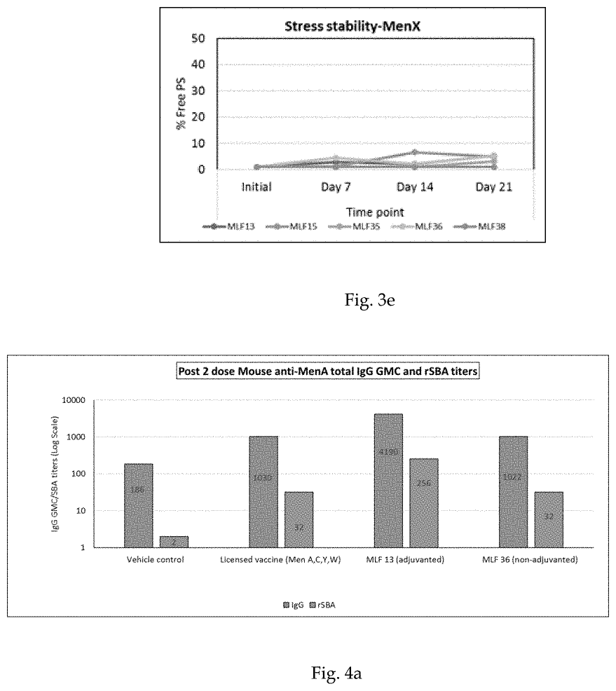

c. stress conditions at 37±2° C.—extreme conditions (Stress Stability Studies) (FIG. 3 a-e).

Samples have been stored for a period of 21 days under stress conditions, for 6 months under accelerated conditions and for 3 years at real time storage conditions (Study ongoing, data available till 9 months). Samples have been withdrawn as per plan and analyzed for the best stability indicating parameter of free PS % content over time.

### Example 10: Immunization of Mice and Rabbits with the Liquid Pentavalent Meningococcal ACYWX-TT Conjugate Vaccine Formulation

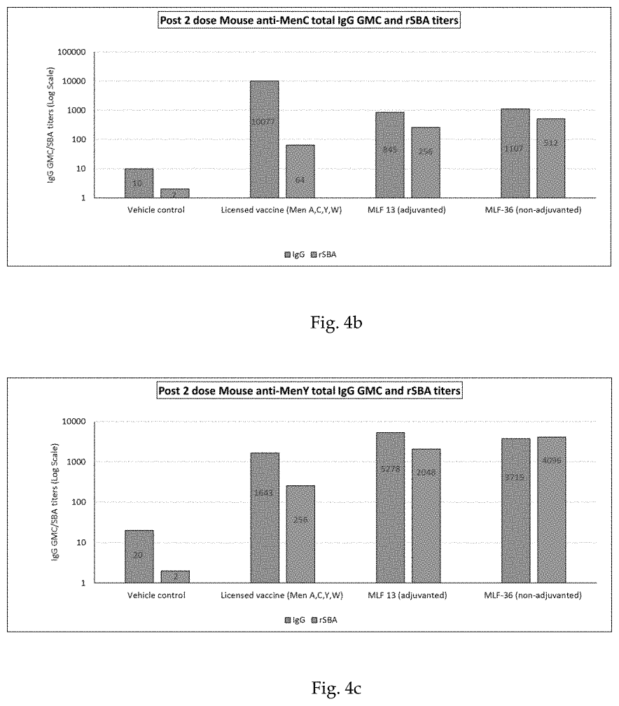

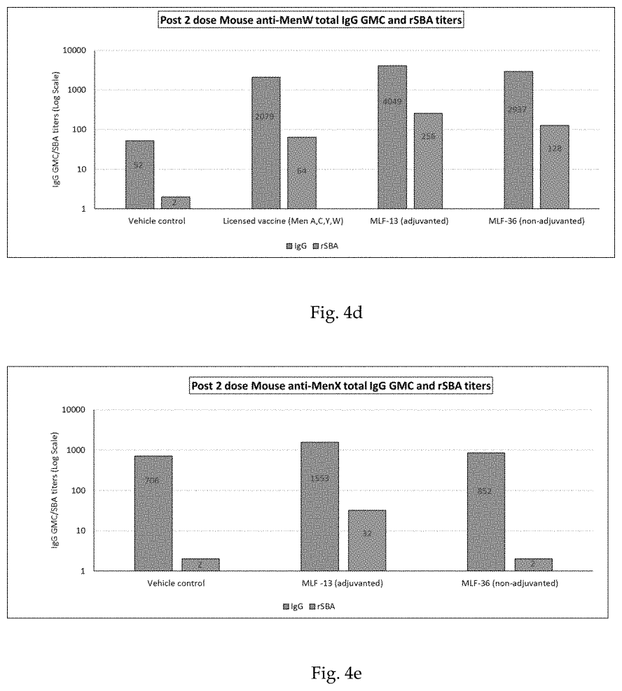

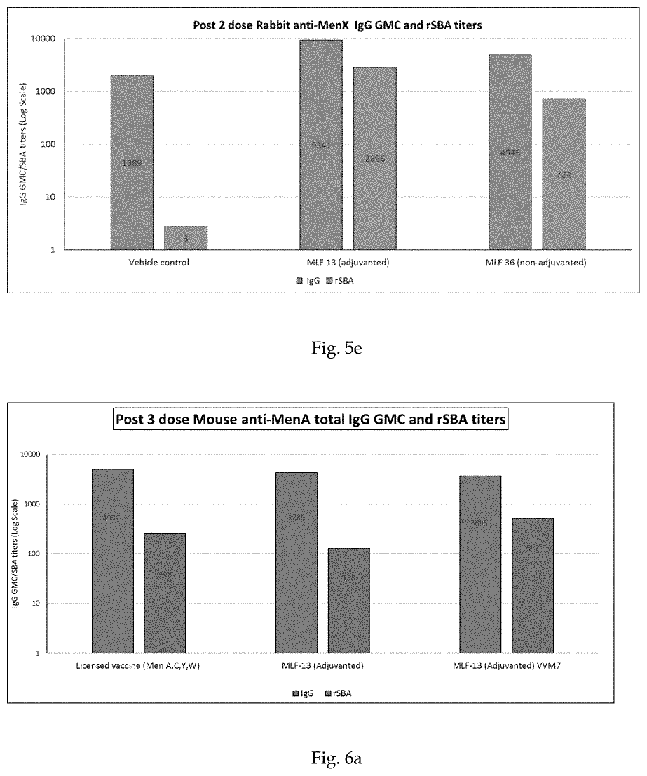

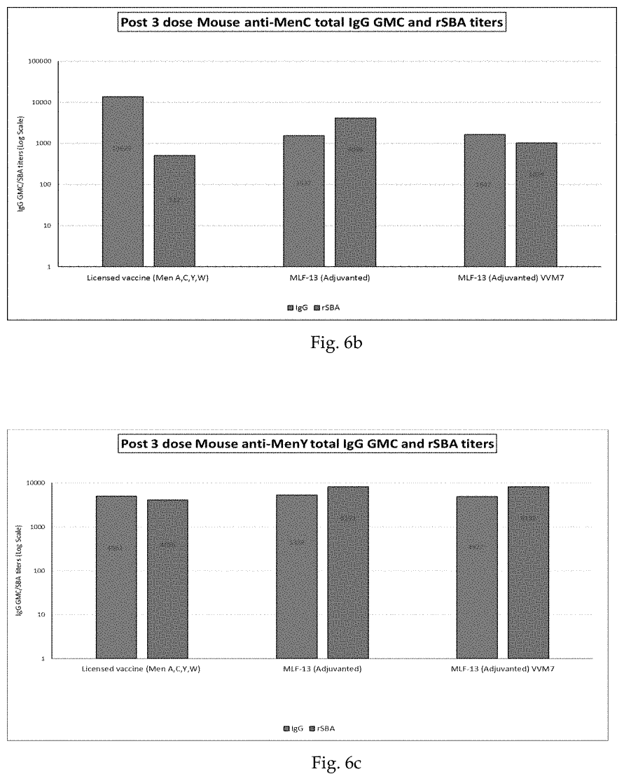

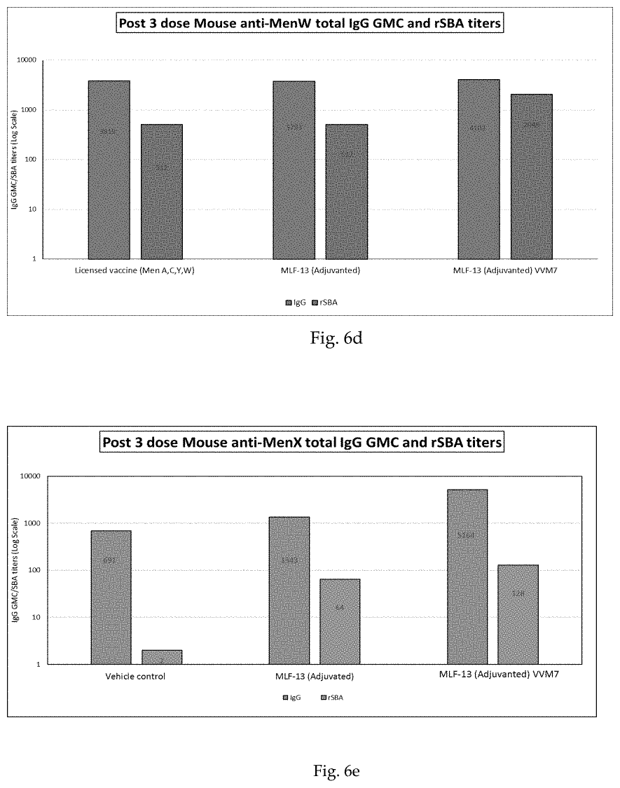

Groups of 8 female mice and 4 female rabbits (6-9 weeks old) have been immunized at 2 week intervals with either novel liquid adjuvanted or non-adjuvanted pentavalent meningococcal ACYWX-TT conjugate vaccine, a vehicle control without bulk conjugates or a licensed ACYW conjugate vaccine. The novel liquid pentavalent meningococcal ACYWX-TT conjugate vaccine has also been used in mouse model after storage at 37° C. for 7 days (VVM7) for evaluation of formulation stability. All immunizations have been performed by administering of vaccine via subcutaneous route in mice and intramuscular route in rabbits. Each mouse has been immunized with formulation equivalent to 1 μg polysaccharide per serogroup, whereas each rabbit has been immunized with full intended human dose. Serogroup specific anti-meningococcal IgG antibody titers have been estimated by indirect ELISA and functional antibody titers by serum bactericidal assay in sera collected post 2 and 3 dose. The post 2 and 3 dose results for novel liquid pentavalent meningococcal ACYWX-TT conjugate vaccine indicate significantly high immunogenicity titers as compared to vehicle control in both mouse and rabbit model and non-inferior titers to the licensed vaccine IgG and SBA titers in both animal models (FIG. 4a-4e, 5a-5e, 6a-6e). Further, the novel liquid pentavalent Meningococcal ACYWX-TT conjugate vaccine showed no decrease in immunogenicity titers when exposed to 37° C. for 7 days (FIG. 6a-6e).

### Example 11: Determination of Anti-Meningococcal Polysaccharide Serogroup Specific IgG Titers by Indirect ELISA

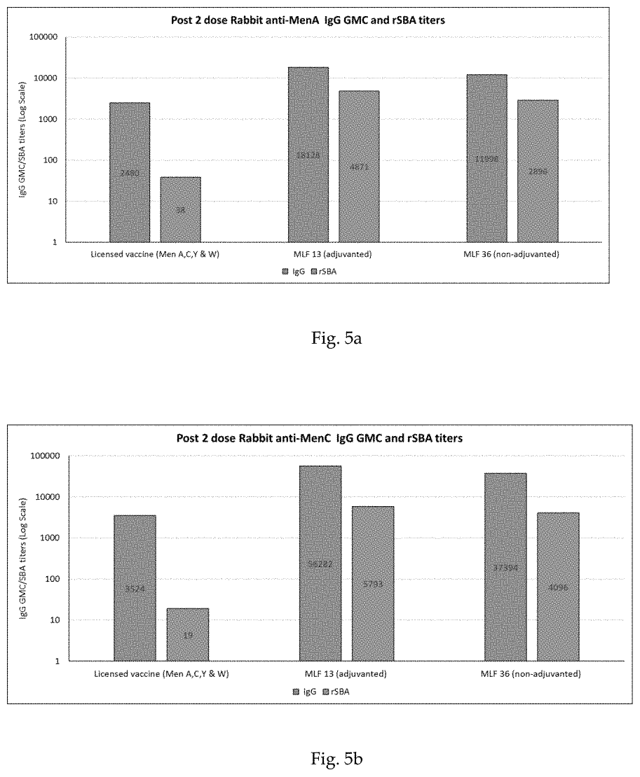

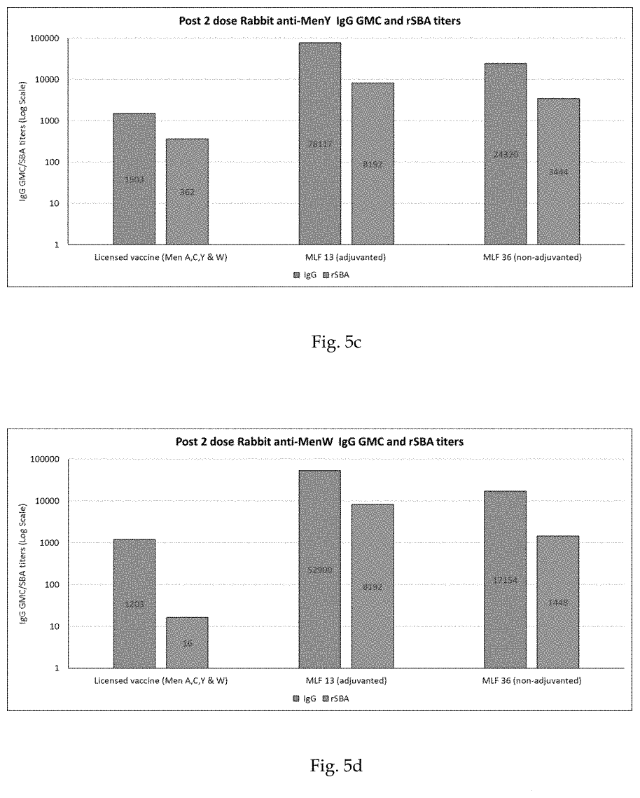

Ninety six-well plates (Nunc Maxisorp) have been coated with serogroup specific standard Meningococcal PS by adding 100 μl per well mixture of a 5 μg/ml PS and m-HSA in PBS buffer, pH 7.3±0.1. Plates have been incubated overnight at 4° C., and then washed three times with PBS buffer (0.1% Brij 35 in PBS, pH 7.3±0.1) and blocked with 200 μl per well of 5% FBS solution in PBS buffer (0.1% Brij 35 in PBS, pH 7.3±0.1) for 1 hour at 37° C. Each incubation step has been followed by three PBS buffer wash. Reference and test sera samples have been diluted in PBS buffer (0.1% Brij 35, 5% FBS in PBS, pH 7.3±0.1), transferred into coated-blocked plates (200 μl), and serially twofold diluted followed by overnight incubation at 4° C. Then 100 μl per well of optimally diluted peroxidase conjugated anti-mouse/rabbit IgG have been added and left for 1 hour at 25° C. 100 _{I}A per well of substrate, 3, 3′, 5, 5′—tetramethylbenzidine-H₂O₂ has been added for color development. After 10 minutes of development at 25° C., reaction has been stopped by adding 50 μl of 2 M H₂SO₄, and OD has been measured at 450 nm on Micro plate reader. Anti-MenC polysaccharide IgG concentrations (in terms of ELISA Units/ml) for each formulation have been evaluated using Combistat software and the geometric mean concentrations (IgG GMC) have been shown for representative studies and formulation comparisons in FIG. 4-6 (FIG. 4a-e 5a-e, 6a-e).

### Example 12: Serum Bactericidal Assay (SBA) for the Serogroup Specific Functional Antibody Titration

N. meningitidis serogroup specific bacterial stock has been grown overnight on sheep blood agar plate at 37° C. with 5% CO₂. Isolated colonies have been picked and incubated on the surface of another sheep blood agar plate at 37° C. with 5% CO₂. The bacterial growth from second plate have been suspended in optimized SBA buffer for respective serogroup. The optical density (OD₆₅₀) of the suspension has been adjusted in working bacterial stock to achieve a colony count of 60-250 per spot in the end of the assay. Quality control (QC) sera and test sera samples have been heat inactivated for 30 min at 56° C. In micro well plate, 20 μl of serial two-fold dilutions of test serum has been mixed with 10 μl of bacteria at the working dilution and 10 μl of baby rabbit complement (Pel-Freez). For negative controls bacteria have been incubated, in a separate well, with active baby rabbit complement without the test serum and with test serum and heat-inactivated baby rabbit complement. The well contents have been mixed by gently tapping the assay plate and incubated the plates for 1 hour at 37° C. with 5% CO₂. Ten μL sample from each well plated on blood agar plate by streak plate method. The blood agar plates have been incubated overnight at 37° C. with 5% CO₂ and colonies have been counted. The highest serum dilution showing ≥50% decrease in colony-forming units after incubation of bacteria with reaction mixture, as compared to respective active complement control has been considered as the SBA titer. The results for representative studies and formulation comparisons are presented in FIG. 4-6 (FIG. 4a-e 5a-e, 6a-e).

1. Novel multivalent polysaccharide-protein conjugate vaccine formulation wherein said formulation is a liquid or lyophilized or a Liquid-Lyo combination pentavalent formulation of Neisseria meningitidis serogroup A, C, Y, W and X capsular polysaccharides (Men A, C, Y, W, X), each said polysaccharide being conjugated separately to tetanus toxoid (TT) carrier protein to obtain Men A, C, Y, W, X-TT conjugates, with one or more buffer and with or without an adjuvant along with pharmaceutically acceptable components/excipients wherein said formulation provides desired osmolality, desired pH, high stability and desired immunogenicity.

2. The novel vaccine formulation as claimed in claim 1 wherein said liquid formulation comprises of:

| Ingredient | Quantity/Concentration |
|---|---|
| Men A, C, Y, W, X -TT | 4-20 μg polysaccharide/serogroup/ml |
| Buffer | 5-30 mM Phosphate buffered saline |
| Excipient | 0-150 mM NaCl |
| | 5-30 mM Histidine |
| Adjuvant: | Aluminum phosphate as 500-2000 μg Al⁺⁺⁺/ml |
| Water (MQW) | qs |

3. The novel vaccine formulation as claimed in claim 1 wherein said liquid formulation comprises of:

| Ingredient | Quantity/Concentration |
|---|---|
| Men A, C, Y, W, X -TT | 10-20 μg polysaccharide/serogroup/ml |
| Buffer | 10-25 mM Phosphate buffered saline (pH |
| | 7.0 ± 0.2) |
| Excipient | 0-150 mM NaCl |
| Adjuvant: | Aluminum phosphate as 750-1500 μgAl⁺⁺⁺/ml |
| Water (MQW) | qs |

4. The novel vaccine formulation as claimed in claim 1 wherein said liquid formulation comprises of:

| Ingredient | Quantity/Concentration |
|---|---|
| Men A, C, Y, W, X -TT | 10-20 μg polysaccharide/serogroup/ml |
| Buffer: | 10 mM PBS (pH 7.0 ± 0.2) |
| Excipient: | 0-150 mM NaCl |
| Adjuvant | Aluminum phosphate as 1000 μgAl⁺⁺⁺/ml |
| Water (MQW) | qs |

5. The novel vaccine formulation as claimed in claim 1 wherein said liquid formulation comprises of:

| Ingredient | Quantity/Concentration |
|---|---|
| Men A, C, Y, W, X -TT | 10 μg polysaccharide/serogroup/ml |
| Buffer: | 10 mM PBS (pH 7.0 ± 0.2) |
| Excipients: | 0-150 mM NaCl |
| Water (MQW) | qs |

6. The novel vaccine formulation as claimed in claim 1 wherein said lyophilized formulation comprises of:

| Ingredient | Quantity/Concentration |
|---|---|
| Men A, C, Y, W, X -TT | 10-20 μg polysaccharide/serogroup/ml |
| Buffer: | 5-20 mM PBS (pH 6.5-7.5) |
| Excipients: | 2-10% |
| Diluent | qs |

7. The novel vaccine formulation as claimed in claim 1 wherein said Liquid-Lyo combination formulation comprises of:

| Ingredient | Quantity/Concentration |
|---|---|
| Men A, C, Y, W, X -TT | 10-20 μg polysaccharide/serogroup/ml |
| Buffer: | 5-20 mM PBS (pH 6.5-7.5) |
| Excipients: | 2-10% |
| Diluent | qs |

8. The novel vaccine formulation as claimed in claim 1 wherein said Men A, C, Y, W, X-TT conjugates are obtained employing optimized combination of conjugation chemistry.

9. The novel vaccine formulation as claimed in claim 8 wherein said conjugation chemistry to obtain said at least one conjugate is carbamate chemistry.

10. The novel vaccine formulation as claimed in claim 9 wherein said at least one conjugate employing said carbamate chemistry is preferably MenX-TT conjugate.

11. The novel vaccine formulation as claimed in claim 8 wherein said conjugation chemistry to obtain said at least one conjugate is cyanylation chemistry.

12. The novel vaccine formulation as claimed in claim 11 wherein said at least one conjugate employing said cyanylation chemistry is preferably said Men A, C, Y, W-TT conjugates.

13. The novel vaccine formulation as claimed in claim 1 wherein each said capsular polysaccharide is degraded in the size range of 0.38±0.1 Kd when tested for molecular size distribution using HPLC PWXL4000 and 5000 columns in series, preferably in the size range of 0.38±0.06 Kd to obtain conjugates with high antigenicity, high immunogenicity and high stability.

14. The novel vaccine formulation as claimed in claim 1 wherein said conjugates are produced having linker arm between said polysaccharide and said carrier protein wherein linker is attached to either said polysaccharide or said carrier protein or both the said polysaccharide and carrier protein.

15. The novel vaccine formulation as claimed in claim 14 wherein said linker is selected from adipic acid dihydrazide or hydrazine.

16. The novel vaccine formulation as claimed in claim 1 wherein said MenX-TT conjugate preferably has hydrazine linker and said MenA, C, Y, W-TT conjugates preferably have adipic acid dihydrazide linker.

17. The novel vaccine formulation as claimed in claim 1 wherein each said conjugate has the carrier protein to polysaccharide ratio between 0.3-1.0.

18. The novel vaccine formulation as claimed in claim 1 wherein said pharmaceutically acceptable excipients are selected from adjuvant, buffer, preservative, stabilizer, surfactant, either alone or in combination.

19. The novel vaccine formulation as claimed in claim 6 wherein said excipient is selected from Sucrose, Maltose, Arginine, Lactose, Sorbitol, Histidine, Glycine, either alone or in variable combinations.

20. The novel vaccine formulation as claimed in claim 6 wherein said diluent is selected from water, 5-20 mM phosphate buffered saline, aluminum phosphate as 500-1500 μg Al⁺⁺⁺/ml either alone or in variable combinations.

21. The novel vaccine formulation as claimed in claim 7 wherein said lyophilized (lyo) portion contains MenA-TT conjugate, MenC-TT conjugate either alone or in variable combinations.

22. The novel vaccine formulation as claimed in claim 7 wherein said excipient is selected from Sucrose, Maltose, Arginine, Lactose, Sorbitol, Histidine, Glycine, either alone or in variable combinations.

23. The novel vaccine formulation as claimed in claim 7 wherein said diluent is selected from water, 5-20 mM phosphate buffered saline, aluminum phosphate as 500-1500 μg Al⁺⁺⁺/ml containing MenC-TT, MenY-TT, MenX-TT, MenW-TT either alone or in variable combinations.

24. The novel vaccine formulation as claimed in claim 1 wherein said formulation is liquid or lyophilized formulation or a combination thereof with mono- or multi-dose regimen with or without a preservative.

25. The novel vaccine formulation as claimed in claim 1 wherein optimum human dose of serogroups A, C, Y, W and X ranges between 2-10 μg polysaccharide per serogroup per dose, preferably 5-10 μg each of serogroup A and X polysaccharides and 5 μg each of serogroup C, Y and W polysaccharides..

26. The novel vaccine formulation as claimed in claim 1 wherein said desired osmolality of formulation is 240-330 mOsmol/Kg.

27. The novel vaccine formulation as claimed in claim 1 wherein said desired pH of formulation is 6.5-7.5.

28. The novel vaccine formulation as claimed in claim 1 wherein said lyophilized portion of the lyophilized or liquid-lyo formulation has a moisture content not more than 3%.

29. The novel vaccine formulation as claimed in claim 1 wherein said vaccine formulation is stable at high temperature of 37±2° C. for at least 21 days and at 25±2° C. for at least 3 months showing less than 40% free polysaccharide.

30. The novel vaccine formulation as claimed in claim 1 wherein said vaccine formulation shows high antigenicity and high immunogenicity.

31. The novel vaccine formulation as claimed in claim 1 wherein said vaccine formulation is equally immunogenic even after exposure at 37±2° C. for 7 days as compared to the formulation stored at real-time storage conditions.

32. The novel vaccine formulation as claimed in claim 1 wherein said formulation is preferably a liquid pentavalent meningococcal conjugate vaccine formulation with mono- or multi-dose regimen with or without a preservative.

33. The novel vaccine formulation as claimed in claim 1 wherein said formulation and said composition is capable of being used in production of liquid or lyophilized or liquid-lyo pentavalent meningococcal ACYWX-TT combination vaccine.
